(19) United States
(12) Patent Application Publication
(10) Pub. No.: US 2014/0372045 A1
(43) Pub. Date: Dec. 18, 2014

(54) Method and an apparatus for indirect measurement of fluid in a container and communication thereof

(71) Applicants: Panu Matti Keski-Pukkila, Helsinki (FI); Yrjoe Tapani Honkavaara, Helsinki (FI); Heikki Matti Tapani Saeteri, Espoo (FI); Olli Taavetti Tiihonen, Lappeenranta (FI)

(72) Inventors: Panu Matti Keski-Pukkila, Helsinki (FI); Yrjoe Tapani Honkavaara, Helsinki (FI); Heikki Matti Tapani Saeteri, Espoo (FI); Olli Taavetti Tiihonen, Lappeenranta (FI)

(21) Appl. No.: 14/307,337

(22) Filed: Jun. 17, 2014

Related U.S. Application Data
(60) Provisional application No. 61/835,671, filed on Jun. 17, 2013.

(51) Int. Cl. A61B 5/00; G01F 23/00; G01P 15/00; G01B 21/20; G01B 21/22

(52) U.S. Cl. CPC A61B 5/4875; G01B 21/20; G01B 21/22; G01P 15/00; G01F 23/00
USPC 702/19

(57) ABSTRACT

A fluid intake monitoring system comprises a sensor band and a mobile device. The sensor band is configured for selective attachment to a fluid container and includes a microprocessor, an accelerometer, and an RF transceiver. The accelerometer measures and outputs acceleration data indicative of the movement of the fluid container and the microprocessor processes the acceleration data and computes threshold values. The mobile device is configured to wirelessly communicate with the sensor band and has a mobile software application comprising modules for receiving data from the sensor band, computing and distinguishing drinking events and non-drinking events. True drinking events are stored and compared with a baserate of hydration for a particular user. Alerts are sent to the user for optimized level of hydration.

### FIELD OF INVENTION

The present invention relates to measurement devices for measuring fluid intake by users, and more particularly, to devices for measuring and correlating movement of fluid containers with fluid intake by a user.

### BACKGROUND OF THE INVENTION

The trend of the “quantified self” is booming and more and more users want to track and improve different biometrics. Physical activities have been tracked with different sensors for decades (i.e. pedometers), but lately the sensors have become much more sophisticated (i.e. wearable activity sensors such as Nike Fuelband or Jawbone UP). Many bodily functions are nowadays measured using sensors, but one of our most basic functions, hydration, has not been properly addressed. After all, hydration is one of the most important factors contributing to overall health as well as physical and mental performance, and it is time that our solution will be filling this gap.

For directly measuring hydration levels from humans, previous inventions have suggested a sensor based on saliva test (U.S. Pat. No. 8,734,341) and earpiece measuring core body temperature (U.S. Pat. No. 8,574,165) for example. One example of an indirect method for estimating hydration need of an individual is an algorithm-fueled pedometer (U.S. Pat. No. 7,493,232). However, these solutions have not gone through clinical trials, and their reliability can be questioned. A reliable, accurate and effortless method for measuring hydration status directly from the human body has not been developed yet, because measuring external and internal water balance at the cell level has been proven to be an extremely complex task. Future solutions will likely be directed to the medical industry and thus the price range will be out of reach for an average consumer.

A more affordable and simple option for directly tracking hydration levels is to use mathematical formulas to estimate the optimal amount of water needed by an individual, and then simultaneously track their liquid intake. This way it's possible to estimate how well hydrated the individual is at different times. The devices needed for tracking liquid intake don't require approval of medical doctors, and consist of off-the-shelf components, making them suitable for the consumer market.

There are different options for tracking liquid intake. One of these is the “Hydracoach” bottle (U.S. Pat. No. 6,212,959). It measures the water flow from a specific bottle by using a mechanical propeller. As another simple solution, some bottles have printed measure scales that allow the user to visually inspect the liquid level in the container. However, neither Hydracoach nor print-measure bottles are truly digital solutions, or able to transmit the data to another device. Furthermore, the means are bound to a certain liquid container (i.e. bottle) and don't allow the user to track liquid intake from different containers.

Hydration-tracking mobile applications for smartphones are popular, and there are tens of different implementations available for iOS and Android platforms. The mobile apps often have a formula that calculates an optimal water intake amount for the individual based on some physical information such as age and weight. The user has to input water intake amounts in the mobile app manually, which is troublesome and neglected by 98% of the users. Thus, these mobile apps do not provide the value they could.

Heretofore, there is no device for monitoring water intake from a fluid container based on movement of the fluid container.

### SUMMARY OF THE INVENTION

An object of the invention is to monitor fluid intake of a user.

Another object of the invention is to provide a system that is capable of detecting and indirectly measuring fluid intake of a user based on user movement of a fluid container.

According to one embodiment, the solution combines a flexible, affordable and digital sensor band to monitor the water intake from any drinking vessel with a connected companion mobile app. The new sensor band device is more advanced than the present sensor devices and is the first device ever to feature direct wireless communication with a mobile app, removing one of the biggest pain points for the user: manual water intake monitoring. The sensor band can also be effortlessly removed from one container or bottle and attached to another one, regardless of whether the second container or bottle is made of different materials, or of different shape or size.

In another embodiment, the fluid intake monitoring method comprises the steps of receiving, by a computer processor, physical characteristics of the fluid container including its shape and volume; measuring, by an accelerometer, movement of the fluid container; processing acceleration data generated by the accelerometer during movement of the fluid container; determining and monitoring, by the computer processor, a tilt angle of the fluid container during movement of the fluid container based on the acceleration data; classifying the movement as a true or false drinking event based on parameters including the determined tilt angle of the fluid container and duration of the movement; determining an amount of reduction of fluid from the fluid container based on the tilt angle, the duration of the movement, and the physical characteristics of the fluid container when the movement is classified as a true drinking event; and outputting a signal indicative of the amount of reduction of fluid from the fluid container.

In another embodiment, an apparatus for determining fluid intake by a user from a fluid container containing liquid, comprises:
(a) a sensor band configured for selective attachment with the fluid container and to include:
   (i) an accelerometer for generating acceleration data based on movement of the sensor band;
   (ii) a microprocessor for processing the acceleration data and for computing a tilt angle of the fluid container based on the acceleration data; and
   (iii) a wireless transceiver;
(b) a mobile device configured for communicating with the sensor band via the wireless transceiver of the sensor band, including:
   (i) a user interface for receiving personal information and physical characteristics of the fluid container from the user and for outputting data to the user;
   (ii) a classification module for classifying the acceleration data from the accelerometer into drinking and non-drinking events;
   (iii) an estimator module for determining an amount of fluid reduction from the fluid container based on the classified data from the classification module and the physical characteristics of the fluid container; and
   (iv) a water baserate calculation and optimization module for computing a baserate of hydration of the user based on the personal information received from the user interface and comparing the baserate with the amount of fluid reduction determined by the estimator module.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 illustrates an embodiment of a sensor band configured for selective attachment to a fluid container;

FIG. 2 diagrammatically depicts the electronic components of the sensor band of FIG. 1.

FIG. 3 illustrates the various screen displays on a mobile device indicating to the user, among others, the fluid intake data.

FIG. 3a diagrammatically depicts the different modules of a software application on a mobile device.

FIG. 4 illustrates an embodiment of the fluid intake monitoring system in communication with cloud computing servers via the Internet.

FIG. 5 is a flowchart showing the baserate calculation algorithm.

FIG. 6 is a flowchart showing the collection and storage of physical characteristics of the fluid container or bottle.

FIG. 7 is a flowchart showing the steps of calibration of the fluid intake monitoring system.

FIG. 8 is a flowchart showing the fluid intake monitoring algorithm.

FIG. 9 illustrates the 6-step drinking event monitored by the inventive system.

FIG. 10 illustrates an exemplary acceleration curve of a drinking event.

FIG. 11 illustrates the bottle tilt angle calculation.

FIG. 12 depicts an exemplary decision tree for drinking and non-drinking events.

FIG. 13 illustrates an exemplary classification methodology for distinguishing drinking and non-drinking events.

FIG. 14 depicts an exemplary decision boundary.

FIG. 15 illustrates true and false drinking events.

FIG. 16 illustrates the factors influencing the estimated amount of water drank from a fluid container.

FIG. 17 shows the measurement of the duration of drinking event.

FIG. 18 shows the duration of drinking event oscillating around the Drinking Event Duration Threshold.

FIG. 19 shows the calculation of duration of the drinking event from datapoints above the Drinking Event Duration Threshold.

FIG. 20 shows an exemplary regression analysis.

FIG. 21 shows the fitting of an estimator curve to a dataset.

FIG. 22 shows an upwards trend in data towards an empty bottle.

### DETAILED DESCRIPTION OF THE PRESENTLY PREFERRED EMBODIMENTS GLOSSARY

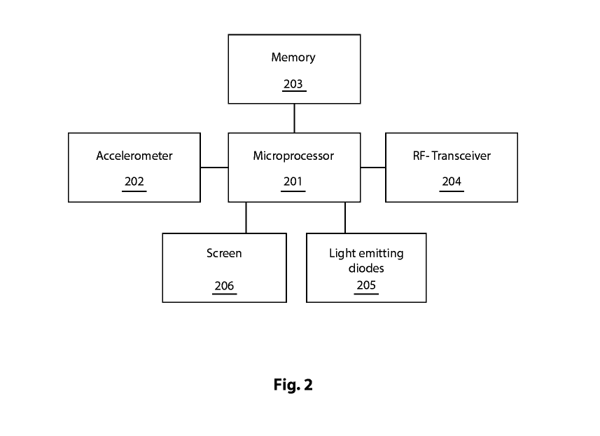

“Wake up threshold” is a static threshold value which is configured in a digital accelerometer 202 (e.g., FreeScale MMA8652FC) (FIG. 2). If the acceleration values exceed the Wake up threshold, the accelerometer sends a wake-up signal to the microprocessor to activate the microprocessor to begin measurement (i.e. the start of a measurement period). In other words, it is used to filter out non-drinking movement and to “wake up” the microprocessor 201 from “sleep mode”.

“Event Threshold” is a static threshold, which is configured in the microprocessor 201 (FIG. 2). The Event Threshold is a first reference tilt angle of the fluid container defining a beginning of a possible drinking event. It marks the start and/or end points of the drinking measurement period and it's the first threshold to check if the current measurement event is a drinking event or a non-drinking event.

“Drinking Event Duration Threshold” is a static threshold, which is also configured in the microprocessor 201. It is a second reference tilt angle for defining a steeper tilt angle for the purpose of reducing the amount of drinking measurement data to include more probable drinking event data and to eliminate probable non-drinking event data.

“Upright Threshold” is a static threshold value of a third reference tilt angle being less than or equal to an upright angle preferably defined as 20 degrees from the gravity vector. It is also configured in the microprocessor 201. The Upright Threshold is compared with a historical record of the bottle's orientations or tilt angles calculated from the acceleration data corresponding to acceleration parallel to the height of the bottle. Simplified, the Upright Threshold requires the bottle to be in an upright position (i.e., meets the Upright Threshold) immediately before such as for example. 1 second before the drinking measurement period can start.

The below describes the presently preferred embodiments of the invention in reference to the attached drawings.

Section 1: Fluid Container and Sensor Band

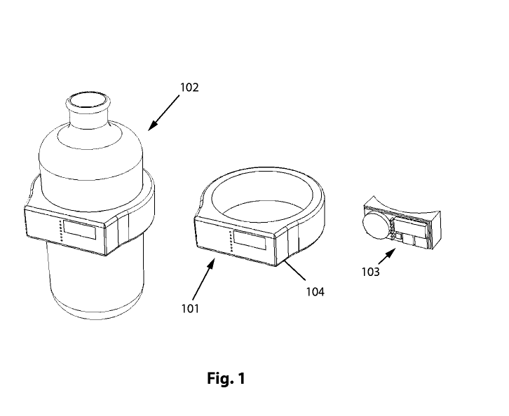

FIG. 1 illustrates an embodiment of the sensor band 101 attached to a liquid container 102. The sensor band 101 includes a housing 104, which covers and protects the electronics 103, The sensor band is designed so that it can be effortlessly attached to a liquid container, and it can be made of materials such as, for example, plastic and/or rubber.

The housing of the sensor band 101 (along with the electronics within) can be attached for example to the liquid container 102 in different ways, one of them being that of slipping a circular shaped casing around the liquid container.

The electronics inside the housing could get their power from a battery, or even possibly from a kinetic device that transforms motion into electrical energy. As shown in FIG. 2, the sensor band 101 has an accelerometer 202 for tracking the motion of the sensor band 101. Using an inventive algorithm as described below, the sensor band 101 recognizes the following information from the data from accelerometer 202:

a) The motion (or process) of the user lifting up the liquid container 102, setting it to his mouth, tilting it to drink and then reversing the same steps to return the container 102 to its somewhat original position.

b) The time that the above-mentioned process takes and the duration between those processes (i.e. lifting up and tilting the container 102 for drinking and returning it to its original storage position).

c) The tilt angle of the container 102, which is used to measure how “full” or “empty” the liquid container 102 is and to identify when measurement should be performed.

d) Activity and non-activity detection to reserve battery life when movement of the liquid container 102 is below a certain specified threshold level.

Section 2: Sensor Band Components

FIG. 2 diagrammatically depicts the various exemplary components of the sensor band 101 comprising a microprocessor 201, an accelerometer (e.g., FreeScale MMA8652FC) 202, a memory 203, an RF transceiver 204, light emitting diodes (“LEDs”) 205, and screen 206. Preferably, these components are placed on the same circuit board. The microprocessor 201 within the sensor band 101 processes the data (as described above) and send it wirelessly using, for example, Bluetooth or RF transceiver 204, to a mobile device or base station (e.g., a smart phone or a wearable device such as a smart watch). In the mobile device, a mobile software application (referred to as the “mobile app” hereafter) may process the data and/or send the data to a private cloud server 402 for processing, storage and possibly later use. The sensor band 101 may use the embedded memory 203 to buffer or store data, and send it over to the mobile device at predetermined intervals in order to, for example, save battery life.

The sensor band 101 may be equipped with a screen 206 and/or other visual/audible indicators, such as LEDs 205 that may communicate the data derivatives (i.e., analyzed data) to the user visually, directly from the sensor band itself. Alternatively, the RF transceiver 204 can also receive commands from a connected smartphone or mobile device, and transfer those commands to the microprocessor 201 to cause an output device such as the LEDs 205 to blink in a predetermined pattern to indicate to the user the status of the fluid measurement. This way the sensor band 101 can be valuable also to users who don't have or who are not used to operating smartphones. One example of these kinds of user groups could be senior citizens or children.

A brief explanation of how to derive drinking data from the accelerometer values in the sensor band:

Identification of the Tilt Angle of the Container:

Preferably, the tilt angle of the container 102 can be calculated, e.g., by positioning the accelerometer 202 so that one of the axes of acceleration is parallel to the container 102 pointing towards or away from the inlet of the container 102. The tilt angle of container 102 can be calculated from the gravity vector. Calculating the tilt angle is not constrained to this positioning of the accelerometer 202 only, since the gravity vector can be calculated using other combination of acceleration axes and is not dependent on the alignment of axes and positioning of the accelerometer 202.

An Exemplary Algorithm:

The algorithm assumes that the amount of liquid drank by a user is proportional to the time the liquid container 102 is tilted and how steep is the tilt angle relative to the gravity vector. The container 102 is probably full or close to full when the tilt angle is small and empty when the tilt angle is steep (e.g., the container 102 is tilted almost upside down). This tilt angle increases due to liquid level decreasing in the container 102. The total time to empty the container 102 can be calculated from the first sip (the most gentle tilt angle) to the last sip (the most steep tilt angle) by adding together the time periods consumed per sip. If this data (i.e., the container tilt angle, the duration of a sip, how many sips per container when empty) is stored we can start comparing new data to old one so that we can get better and more accurate results, since old data might contain information about similar sip tilt angle/length. Not all data needs to be stored and these patterns can be updated accordingly to express more averaged results. Overall, the algorithm will be adapted based on previous data and become more accurate over time as it starts to recognize the drinking patterns of a certain user and his container. It is contemplated that the sensor band 101 and the accelerometer 202 also monitors the oscillatory movement of the bottle 102. Different levels of fluid in the bottle 102 impart different kind of oscillatory movement because of the difference in their mass and inertia. The oscillatory movement can be measured and after processing of the movement data, the analytical results can be used to improve the accuracy of the algorithm using bottle tilt angles and duration and to estimate the amount drank by the user. If the oscillatory movement data helps us define more accurately when the bottle is (close to) full and when it is (close to) empty and we also know the volume of the container, we can adjust the possible error margins related to the algorithm with it. For example, if the algorithm estimates the user drank 3.5 deciliters out of a 5 deciliter bottle but the movement data suggests the bottle is almost empty, the algorithm can be adjusted to show a more accurate reading.

Section 3: User Screen Displays

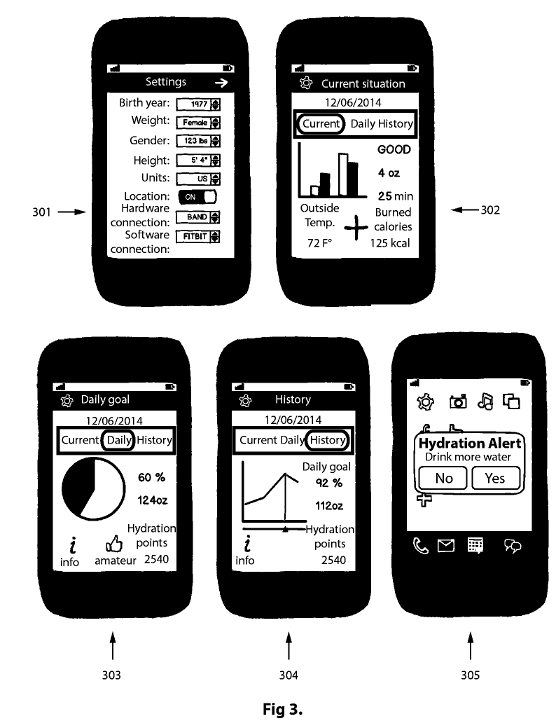

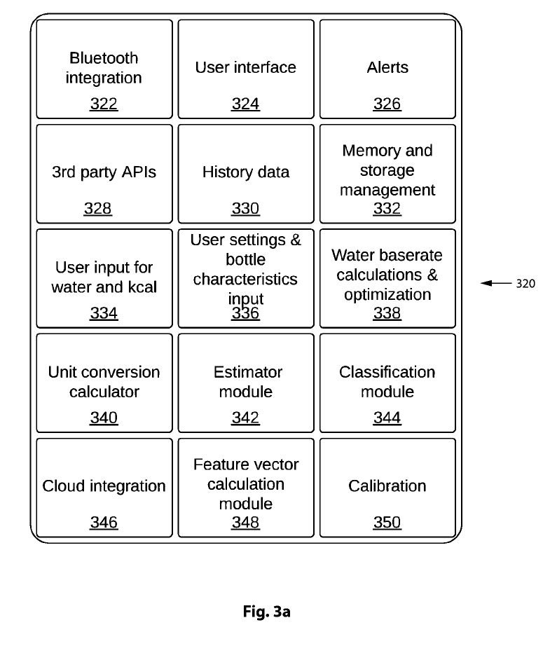

As shown in FIG. 3, the mobile app on a mobile device may consist of the following exemplary screen displays:

Settings screen 301

Current situation screen 302

Daily goal screen 303

Historical screen 304

Push notifications 305

The user may input their “personal information” stats (these could be for example weight, age, height, gender, activity level, eating habits) into the mobile app, and the mobile app may use this data to calculate a “base rate” for balanced hydration as shown in screen 301 for the specific user in question. This base rate may then for example be adjusted according to the environmental conditions (e.g., local temperature and humidity etc.) and the user's physical activity levels. The activity levels may be tracked using GPS data and/or data from activity trackers, for example such as FitBit, Nike Fuelband or Runkeeper Opengraph. By combining the adjusted base rate values with the drinking data from the sensor band, the mobile app knows when to remind the user to drink something to maintain balanced hydration. The user can for example also view their current hydration status at an instance, on a daily basis or as a historical summary. They can also input the drinking data manually as shown by screen 302, if they do not happen to have the sensor band 101.

According to one exemplary feature, the user may be able to share their progress and hydration status in social networks as shown by screen 303, or collect points and “badges” as depicted by screen 303 by keeping up with the balanced hydration schedule.

The above presented an example of how the drink data from the sensor band 101 could be utilized. One could develop different kinds of mobile apps that use the drink data, for example such as one which communicates the environmental impact of the user filling up their own re-usable bottle instead of buying bottled water from the store and gives the user incentives to stick to tap water.

Section 3A: The Applications and Sub-Programs in the Mobile App

FIG. 3A illustrates the structure of the mobile app 320 and the sub-programs or modules within. It is contemplated that the mobile app 320 may also be configured in the sensor band 101 or the mobile device 401 as shown in FIG. 1.

A. Bluetooth integration module (BIM) 322 communicates with the sensor band RF-transceiver 204 by commanding the mobile device RF-transceiver (i.e. a built-in Bluetooth module in the mobile device (not shown)) to send and receive data to the RF-transceiver 204 in the sensor band 101. The data that is being transmitted from RF-transceiver 204 to BIM 322 is drinking event data. The data that is being transferred from BIM 322 to RF-transceiver 204 is alert data (for example, command to blink the LEDs on the band). BIM 322 also sends inquiries (or “pings”) between certain intervals (for example 5 seconds) to RF-transceiver 204 to check if it has new drinking event data. If yes, RF-transceiver 204 sends the drinking event data to BIM 322. If not, sensor band RF-transceiver 204 replies that it has no new data available. UDID (Unique Device ID i.e. device identification code) data is also transferred between BIM 322 and RF-transceiver 204 when the devices are being “paired together. The Bluetooth integration module also interacts with the following modules:

(1) Memory and storage management module 332 (MSMM). This module saves data coming from BIM 322, for example drinking data and UDID. BIM 322 also retrieves data from memory, for example UDID numbers for purposes related to re-identifying previously registered devices.

(2) Alerts Module 326 (AM). Alerts module 326 sends a command through BIM 322 to the sensor band 101 to start blinking LEDs.

B. User interface module (UIM) 324 defines the visualization of the application and the functionality of human-device interaction. FIG. 3 describes examples of different screens the user might see. The User interface represents the parts of the mobile app that the user sees on the screen of their mobile device. UIM 324 interacts with the following modules:

(1) Alerts Module (AM) 326. AM 326 can send commands to UIM 324 to display push notifications alerts.

(2) History data module (HDM) 330. UIM 324 can fetch ask and receive history data from HDM 330, for example to display different past information related to drinking goals or user activity.

(3) User settings & bottle characteristics input module (USBCIM) 336. UIM 324 displays a settings screen in which the user can change settings and input data to. USBCIM 336 defines what data (i.e. age groups, 3rd party APIs that are available, alert settings etc.) will be shown by UIM 324. When the user inputs personal information to UIM 324, the information is sent to USBCIM 336.

(4) User input for water and kcal-module (UIWKM) 334. UIM 324 displays a screen that allows the user to manually input information about the water they drink and exercises they do. UIWKM 334 sends data to UIM 324 that defines what the user sees on that screen. For example, the user might see an array of different types of exercise (e.g., tennis and aerobics) and another array indicating time spent for these exercises. When user chooses an exercise type and time spent, the data is sent to UIWKM 334 that processes it and sends back information about the caloric consumption of the user and shown to the user via UIM 324.

(5) Water baserate calculation and optimization module (WBCOM) 338. WBCOM 338 sends data to be displayed by UIM 324. This data can be for example the user's daily hydration goal, the amount he drank during the day or the amount he needs to drink during the next hour.

C. Alerts module (AM) 326 defines the means to indicate different conditions to the user such as when and how to inform when a user should drink more water. These conditions are detailed more closely in Section 8a below. The Alerts Module 326 has access to hardware in the mobile device which allows, for example, to show push notifications on the device screen or play audible alerts or tunes on the mobile device loudspeaker. Alerts module 326 interacts with the following modules:

(1) Bluetooth integration module 322. Alerts module 326 sends commands via BIM 322 to the sensor band 101 to start blinking LEDs 205, for example.

(2) User interface module 324. See UIM 324 for information about interaction.

(3) User settings & bottle characteristics input module 336. AM 326 receives data from USBCIM 336. This data can include information about the types of alerts the user wants to receive, the interval of the alerts and the times the alerts start and stop (for example start at 7 am and stop at 11 pm). AM 326 sends data to USBCIM 336 to define what options the user has. For example, the user might be able to set the start and stop of the alerts by hours, not minutes.

(4) Water baserate calculation and optimization module 338. WBCOM 338 commands AM 326 to launch alerts.

D. 3rd party APIs module (APIM) 328 communicates with other APIs such as FitBit, Runkeeper and weather API to fetch relevant calorie consumption or outside temperature data. The term API refers to Application Programming Interface, which specifies how some software components should interact with each other. In a nutshell, the APIM 328 module connects to the 3rd party API using the mobile device Internet connection (via cellular network or WiFi). APIM 328 logs in with existing access credentials (which are typically given by the 3rd party API when APIM 328 connects to it for the first time). 3rd party API authenticates the APIM 328 entry and then redirects APIM 328 to the data that it's looking for. In our example, caloric consumption data. APIM 328 fetches the data and saves it to MSMM 332. 3rd party APIs module interacts with the following modules:

(1) Memory and storage management module 332. APIM 328 saves data (kcal consumption, access details, etc) from 3rd party APIs to the memory and also retrieves it when needed. For example when it sends the data to USBCIM 336 or WBCOM 338.

(2) User settings & bottle characteristics input module 336. APIM 328 sends data to USBCIM 336. This data for example defines what 3rd party APIs are available. USBCIM 336 sends data to APIM 328 about what APIs the users wishes to get exercise information.

(3) Water baserate calculation and optimization module 338. APIM 328 sends kcal consumption information to WBCOM 338.

E. History data module (HDM) 330 manages how the past data of drinking events, hydration goals, temperature and activity is visualized, used and stored. For example, the user could view how well they have kept to their hydration goals during the past month and how has that fluctuated according to physical activities i.e. is it hard to keep up to the goals on days the user exercises a lot. This module interacts with the following modules:

(1) User Interface Module 324. See UIM 324 for More Details about Interaction.

(2) Memory and storage management module 332. HDM 330 can fetch history data from MSMM 332 which is stored there by WBCOM 338 (drinking data) or APIM 328 (activity data) for example. HDM 330 can compile graphs and visuals from this data to be sent to UIM 324, and it can also send the graph and visual data back to MSMM 332 for storage.

F. Memory and storage management module (MSMM) 332 controls flow of data and the database storage. This module interacts with the following modules:

(1) Bluetooth integration module 322. See BIM 322 for more details about interaction.

(2) 3rd party APIs module 328. See APIM 328 for more details about interaction.

(3) Water baserate calculation and optimization module 338. See WBCOM 338 for more details about interaction.

(4) Unit conversion calculator (UCC) 340. See UCC 340 for more details about interaction.

(5) Estimator module 342. Stores estimator function and water amount estimates.

(6) Classification module (CM) 344. Stores Classification model and classification results.

(7) Cloud integration (CIM) 346. This module CIM 346 sends and fetches data to and from cloud computing servers and saves and fetches same data from MSMM 332

(8) Feature vector calculation module 348 (FVCM). Stores feature data into MSMM 332.

(9) Calibration module (CAL) 350. Stores Calibration datasets and estimator function into MSMM 332 and retrieves them when needed.

G. User input for water and kcal module (UIWKM) 334. Manual input interface and calculations for manual water and kcal inputs using the user interface of the app. This interface logs information that the user has manually inputted in UIM 324. For example, it calculates kcal expenditure from different combinations of exercise and time. When user chooses an exercise and time (i.e. Tennis for 30 minutes) in UIM 324, the data is sent to UIWKM 334 and it calculates the kcal consumption of the user and returns that to UIM 324 which then displays it to the user. This module UIKWM 334 interacts with the following modules:

(1) User interface module 324. See UIM 324 for more interaction information.

(2) Water baserate calculation and optimization module 338. UIWKM 334 sends data about user manual inputs water and kcal inputs to WBCOM 338.

H. User settings & bottle characteristics input module (USBCIM) 336 provides interface and operations regarding usage and storage of the user's personal settings and bottle characteristics. It handles all the data the user inputs in the mobile app settings, for example their age, gender, weight and unit preferences. This module interacts with the following modules:

(1) User interface module 324. See UIM 324 for more interaction information.

(2) Alerts module 326. See AM 326 for more interaction information.

(3) 3rd party APIs module 328. See APIM 328 for more interaction information.

(4) Memory and storage management module 332. USBCIM 336 sends all the data that it handles (for example alerts, unit preferences and user settings) to MSMM 332. There it's available for other modules needing it, such as WBCOM 338 for example. USBCIM 336 also fetches the previous data from MSMM 332 when needed, for example when the user opens the app and enters the settings screen.

(5) Unit conversion calculator 340. USBCIM 336 interacts with UCC 340 both ways. UCC 340 defines what unit options are available for USBCIM 336. When user changes their preferred unit options, USBCIM 336 sends data to UCC 340 which then converts the units (for example from fluid ounces to litres or from pounds to kilograms) and sends the converted data back to USBCIM 336.

I. Water baserate calculation and optimization module (WBCOM) 338 calculates and updates the baserate and user's water need, based on information from other modules. This module interacts with the following modules:

(1) User interface module 324. See UIM 324 for more details about interaction.

(2) Alerts module 326. See AM 326 for more details about interaction

(3) 3rd party APIs module 328. See APIM 328 for more details about interaction.

(4) Memory and storage management module 332. WBCOM 338 fetches drink amount data from MSMM 332, which has been stored to MSMM 332 by Estimator 342. WBCOM 338 also stores information about base rates and goals to MSMM 332 and fetches it when needed.

(5) User input for water and kcal Module 334. WBCOM 338 receives data from UIWKM 334. The data includes information about the absolute water amounts the user has manually inputted and the kcal expenditure information of manually inputted user activities.

J. Unit conversion calculator 340. See UCC 340 for more details about interaction

(1) Estimator module 342. Estimate for drink amount is received from estimator module.

(2) Unit conversion calculator (UCC) 340. It performs unit conversions, for example from fluid ounces to litres and kilograms to pounds. This module interacts with the following modules:

(3) Memory and storage management module 332. UCC 340 stores its conversion formulas in MSMM 332 and fetches them from MSMM 332 when it needs to perform conversions.

(4) User settings & bottle characteristics input module 336. See USBCIM 336 for more details about interaction.

(5) Water baserate calculation and optimization module 338. WBCOM 338 can send data to UCC 340 for conversion. UCC 340 performs the conversion and sends the converted data back to WBCOM 338. This data is numbers and amounts such as, for example weight or drink amounts.

K. Estimator module (EM) 342 performs estimation of amount drank by user during a drinking event and communicates with water baserate calculations & optimization module 338 to update the consumed water amount. Estimator is formed based on information from the calibration module 350 and the estimator function can be updated or replaced with new one by Calibration Module 350. Estimator Module 342 interacts with the following modules:

(1) Memory and Storage Management 332. Receives event data, estimator and classification results from the MSMM 332 and stores the estimate for consumed water amount into MSMM 332

(2) Calibration Module 350 forms the estimator function, See CAL 350 for more info about interaction.

(3) Water baserate calculation & optimization module 338. See WBCOM 338 for more information about interaction.

(4) Classification module 344. Estimator 342 calculates the water consumption estimate for event data that is classified as drinking event by CM 344.

L. Classification module (CM) 344 receives drink event features from memory and performs a classification into non-drinking and drinking events. Classification model is updated with new events from calibration module 350 to better learn the user's personal drinking events. Classification Module 344 interacts with the following modules:

(1) Memory and Storage Management 332. Receives drink event features from the memory and stores the classification result.

(2) Feature vector calculation module 348. Classification module fetches feature data from FVCM 348 for the classification process.

(3) Calibration module (CAL) 350. Calibration process uses data from the classification module 344 to classify drinking events. Classification module 350 uses calibration drinking event to improve the performance of the classification process.

(4) Estimator module 342. Estimator 342 calculates estimated water drank during drinking event for those events that are classified as a drinking event by the classification module 344.

M. Cloud Integration Module (CIM) 346 communicates with cloud services and memory and storage management 332. All the data stored to memory is stored also in the cloud and can be accessed by the other modules via the memory and storage management 332 to communicate with Cloud Integration Module 346.

N. Feature Vector Calculation Module (FVCM) 348 calculates statistical properties of the drinking event data such as the average value and the standard deviation, and stores them to memory for later use. This module interacts with the following modules:

(1) Memory and storage management module 332. Feature vector calculation module receives data events and stores features of events into MSMM 332. FVCM 348 also receives data (i.e. drinking data) from MSMM 332 that has been stored there by BIM 322.

(2) Calibration module (CAL) 350. Calibration process uses data from FVCM 348 to calculate features of drinking events.

(3) Classification Module (CM) 344 uses feature data from FVCM 348 for its classification calculations and operations.

O. Calibration module (CAL) 350 performs a calibration setup during which the user receives instructions on how to proceed to ensure that the collected calibration dataset is well defined (number of drinking and non-drinking events and their location in collected dataset). Calibration dataset is used for building estimator 342 and improving classification performance. Calibration module performs regression modelling to find a best fit for given calibration dataset forming an estimator 342 function. This module interacts with the following modules:

(1) User interface 324. Calibration Module 350 sends data to User Interface 324 so that it can prompt the user to start and end the calibration process.

(2) User settings & bottle characteristics input 336. Calibration Module 350 uses bottle characteristics data from (UIWKM) 336 to build suitable estimator for the bottle used.

(3) Feature vector calculation module 348 & Classification module 344. Calibration module uses FVCM 348 and CM 344 to classify the calibration data. Calibration module 344 stores the calibration event data which the classification module 344 can use to improve the performance of classification.

(4) Memory and storage management (MSMM) 332. Event information is fetched from the storage and calibration events and estimator function is stored to MSMM 332.

(5) Estimator module (EM) 342. Calibration module builds the parts of the estimator function that Estimator Module uses.

Section 4: Overall Sensor Band/Mobile App Solution

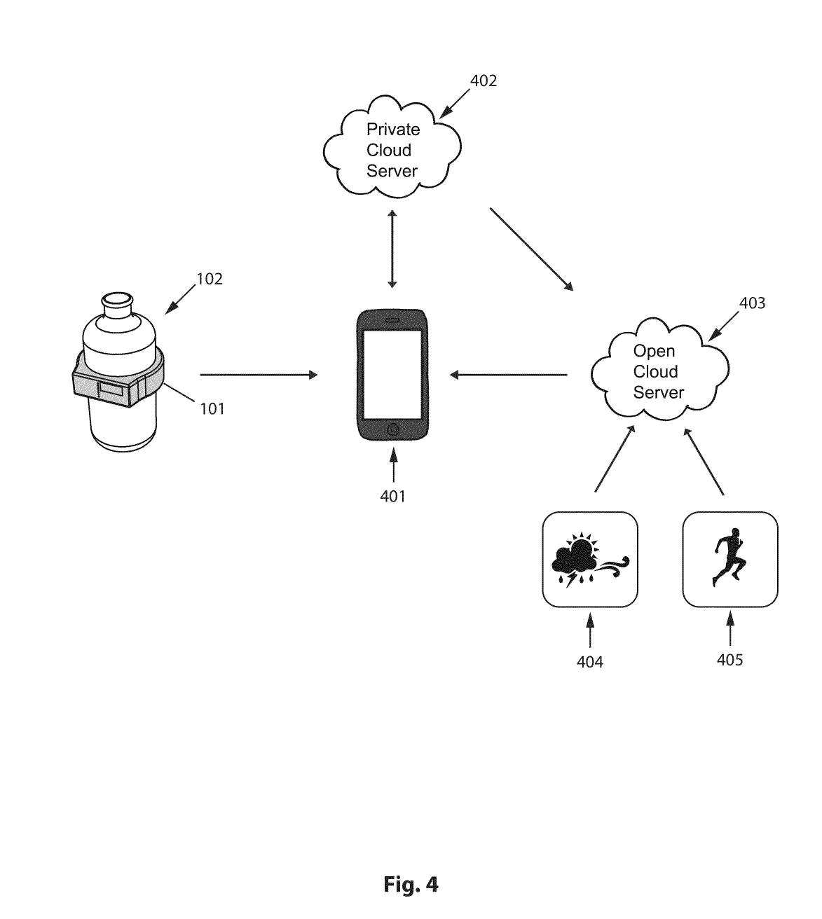

As shown in FIG. 4, an overall sensor band and mobile app solution may be configured to include a mobile app 320 in a mobile device 401, a private cloud server 402, the sensor band 101, an open cloud server 403 with a weather API 404 and an activity API 405 such as, for example FitBit™ or RunKeeper Opengraph™).

The sensor band 102 may send the drink measurement data to a mobile app in the mobile device 401, which may perform hydration coaching calculations based on, for example, user-inputted stats, weather information and activity data. A portion of this information may be inputted by the user manually, and part of it may be derived from open cloud server APIs 404 and/or 405. The mobile app 320 may store part of the hydration information in its own memory or send it to the private cloud server 402 for storage.

Section 5: The Hydration Baserate

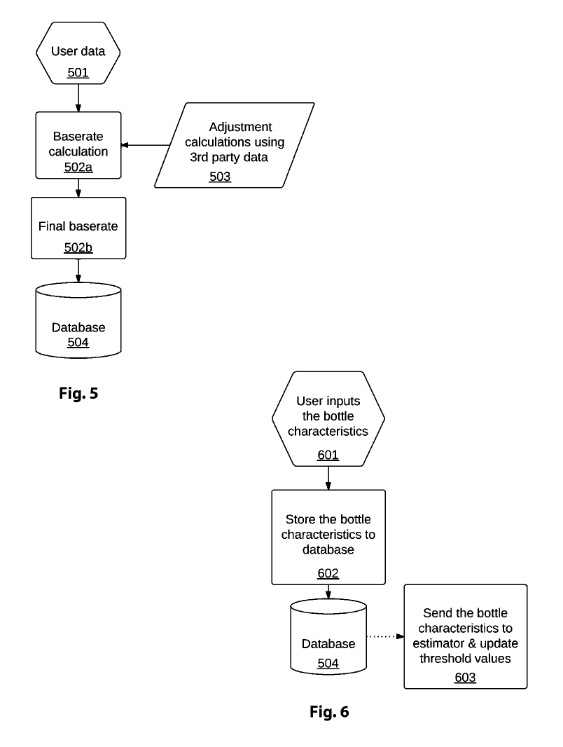

FIG. 5 illustrates the process of determining, calculating and adjusting hydration baserate for a user by a mobile app on a mobile device. The calculated hydration baserate is a reference fluid replacement rate for the user. It may vary based on the user's physical condition, physical activity level and the user's surrounding environmental conditions (e.g., temperature). In general, the amount of hydration required by an individual is proportional to the weight of the individual, and decreases by age. The gender option (male/female) does not affect the computation for optimal hydration amount, unless the user chooses options for female/pregnant or female/breastfeeding. These options add +300 ml/day and +700 ml/day into the BaseRate, respectively.

In Step 501: A user inputs his personal information in the mobile app including (but not limited to), for example, weight, age, and gender.

In Step 502a: The mobile app 320 calculates a baserate for optimal hydration amount using the personal information the user input in Step 501.

BaseRate=The amount of water needed for a person of a certain age and a certain weight. If the user chooses female/pregnant or female/breastfeeding options, the 300 ml/day or 700 ml/day will be added to the BaseRate.

BaseRate (in fl. oz):
Age from 0 to 30->BaseRate=Weight (in lbs)*0.45
Age from 31 to 65->BaseRate=Weight (in lbs)*0.40
Age more than 65->BaseRate=Weight (in lbs)*0.35

+300 ml or 700 ml (in case the user is pregnant or breastfeeding)

BaseRateWOFood=Baserate without food i.e. the BaseRate amount minus the amount of water an average person gets from food daily. Essentially this is the amount that the person needs to drink water in liquid form throughout the day. We assume that an average person eats pretty much the same amount during the day even though the ambient temperature is higher or they do some exercise, and that is why BaseRateWOFood is calculated directly from BaseRate. The temperature and activity adjustments are calculated on top of BaseRateWOFood, because we assume that people mostly drink, rather than eat, to make up for the liquid they have lost in heat and/or exercise.

BaseRateWOFood=BaseRate*0.78

Average person gets 22% of their daily water from food so 100%−22%=78%.

In Step 503, the mobile app links with third-party devices and apps to receive user physical activity and ambient temperature data and performs calculations to arrive at the correct baserate adjustment number. Alternatively, the user may manually input the type and duration of their physical exercise, and the mobile app adjusts the baserate accordingly. Examples of third-party mobile apps include the running tracker called “Runkeeper” or the cycling tracker “Strava”. An example of a third-party device is the activity-tracking bracelet “FitBit”. The baserate calculation in Step 502a is not necessarily the final baserate amount. In case third-party devices and apps provide data that impacts the baserate, the final baserate in 502b is different from the baserate in 502a. In case there is no third-party data, the final baserate in 502b is identical to the baserate in 502a.

The activity adjustment is based on kcal consumption data from the third-party devices. It's been scientifically proven that for each kcal a person expends, the person should ingest or drink about 1 ml of water to maintain proper hydration balance. The 1 mL of water per kcal constant is based on this article at http://www.who.int/water_sanitation_health/dwq/nutrientschap3.pdf by The Center for Human Nutrition.

WaterkcalTotal=WaterkcalFitBit+WaterkcalRunkeeper+WaterkcalOther+WaterkcalManual

WaterkcalTotal is the additional amount of water needed due to the physical activities of the user. Each kcal the user burns equals to 1 ml of additional water they need to drink. The “total” in this formula means that it adds kcal from all of the devices and apps the user has linked. At the moment, our solution has only FitBit device and Runkeeper app linkage, but the list is not limited to these only. The WaterkcalOther represents the additional apps/devices to be linked in the future.

BaseRateLitre=BaseRate/0.033814 (BaseRateLitre is BaseRate (oz) turned to litres. So for example BaseRateLitre=65 oz/0.033814->about 1920 ml (1.92 litres).

WaterkcalFitBit (in fl. oz)=(AllkcalFromFitBit−BaseRateLitre)*0.033814

The WaterkcalFitBit figure consists of the kcal consumption derived from the FitBit API. For example, let's assume a user gets 2500 kcal consumption data from Fitbit API and the mobile app has determined that their BaseRate is 2000 ml (2 litres). Thus, WatercalFitBit=2500−2000->500 ml. The user needs to drink 500 ml more because of their activities. In the last step the 500 ml is converted to fl. oz with the coefficient 0.033814.

WaterkcalRunkeeper (in fl. oz)=RunkeeperExerciseCal*0.033814

This figure consists of the kcal consumption derived from the Runkeeper API. RunkeeperExerciseCal is the amount of kcal burned when the user goes for a run and tracks it with the Runkeeper app. The Runkeeper kcal data is available through their open API (Application Programming Interface, allows software programs to interact with each other), which means our mobile app can fetch it when needed.

WaterkcalOther=This figure consists of the kcal consumption derived from other third party applications and/or devices

WaterkcalManual=This figure consists of kcal consumption of the activities that the user has manually inputted in the mobile application. Our mobile app has manual input for activities. If you don't have a Runkeeper app or Fitbit device, you can manually add exercise, for example a 30 min Spinning workout. Then our app calculates average kcal consumption for the specific time and type of exercise, and calculates how much more water you'd need to drink. The need for water is also adjusted according to ambient temperature. The temperature constants are adapted from ideas presented in the article (http://hprc-online.org/nutrition/files/current-u-s-military-fluid-replacement) by the United States Army Research Insitutute for Environmental Medicine. Again, the number 0.033814 is the universal milliliter (mL) to fluid ounce (fl. oz) conversion constant.

WaterTemp=This is the additional amount of water needed due to the ambient temperature. In our formula, the need for additional water is 2% of BaseRateWOFood with every Celsius degree over 21 degrees.

Celsius 21: WaterTemp=(BaseRate*1.02)−BaseRate

Celsius 22: WaterTemp=(BaseRate*1.04)−BaseRate

Celsius 23: WaterTemp=(BaseRate*1.06)−BaseRate

Celsius 35: WaterTemp=(BaseRate*1.30)−Baserate

Etc.

So for example BaseRate is 100 oz: (100 oz*1.02)−100 oz->You have to drink 2 fl. oz more due to higher ambient temperature.

In this manner, the hydration baserate based on personal information, activity, and temperature is finally adjusted for the user. FinalBaseRate represents the figure user needs to drink during a day to stay properly hydrated. It is also referred to as their hydration goal.

FinalBaseRate=BaseRateWOFood+WaterkcalTotal+WaterTemp

Step 504: The adjusted final baserate is transmitted to the database 504 for storage, and for later comparison with measured fluid intake by the user.

Section 6: Drink Container Characteristics

FIG. 6 illustrates the process of inputting drink container characteristics.

Step 601: User inputs information of their preferred fluid container characteristics, e.g., choosing a bottle type in the app menu from predefined bottle types. The bottle types have predefined characteristics, which include for example, but not limited to container volume, bottle shape and bottle neck shape (e.g., different bottles have different neck shapes). All these characteristics affect the drinking event determination such as the smallest bottle angle that allows drinking from the bottle. Please note that throughout this application, the term “bottle” is not limited to just bottles (i.e. a container with a neck narrower than the body), but it can also refer to other vessels that users might drink from, such as glasses, jars, mugs or cups. These containers may or may not have a cap that can be used to seal the liquid inside.

Step 602: The fluid container characteristics are stored to database 504.

Step 603: Fluid container characteristics are used by the Estimator 342. For example, the container volume is used in calibration phase. The threshold values which define non-drinking and drinking events can be changed regarding the container characteristics. For example, a glass has different characteristics than a bottle. Typically, to drink from the glass the user would need to tilt the glass less than with the water bottle when both are full. Thus, for drinking from a glass, the sensor band needs to be more sensitive for small tilt angles.

Section 7: Calibration and Building the Estimator

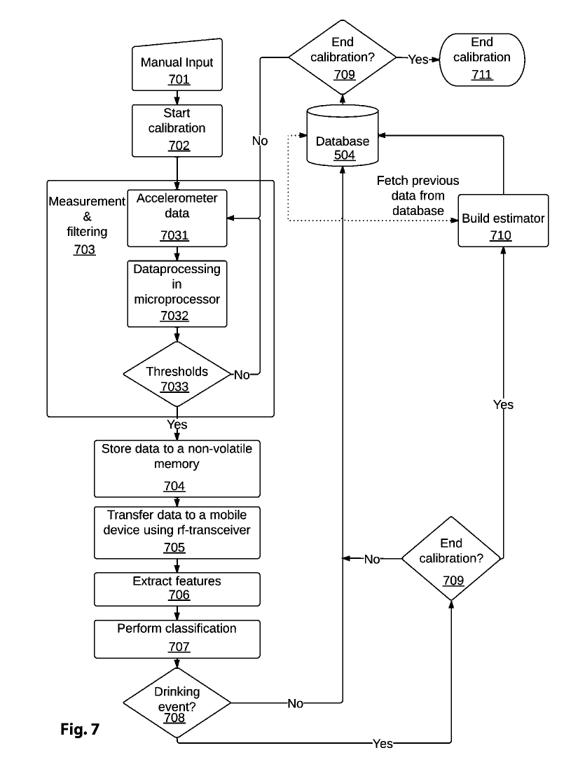

FIG. 7 illustrates the process of calibrating the sensor band 101 and building Estimator 342.

Step 701: User initiates the sensor band calibration according to instructions in the app, e.g., tilting the container or bottle 102 (with the sensor band 101 attached) upside down for a specified time, e.g., 5 seconds assuming the bottle has a cap for sealing, or alternatively by using the manual user inputs in the mobile app to indicate the start and end of calibration with start and end buttons.

Step 702: Sensor band calibration starts. At this point the calibration process retrieves and utilizes the container characteristics inputted by the user and saved in database 504.

Step 703: This step refers to the measurement and filtering process during which movement data is recorded by accelerometer 202, movement data is analyzed in microprocessor 201 and threshold decision made in microprocessor 201. In more detail, the user drinks from the bottle 102 as they would normally do. Every time the bottle moves, the accelerometer 202 in the sensor band 101 detects the movement and sends the data to the microprocessor 201. In the calibration phase, it is assumed that the user does not move the bottle around unless they drink from it, but it is important to have the threshold calculation in step 7033 to take into account of the bottle tipping over by accident or otherwise experiencing movements unrelated to actual drinking. The threshold calculation step 7033 determines whether the movement is a drinking event or not by comparing movement data values from accelerometer, the static threshold values (as described below) and a historical record of various user-induced orientations of the bottle 102. The static threshold values (e.g., Wake Up Threshold, Upright Threshold, Event Threshold, and Drinking Event Duration Threshold) are compared to low pass filtered DC signal values (i.e. acceleration data from the accelerometer 202) which correspond to the tilt angle of the bottle 102. If No, the process starts over from data recording by accelerometer 202. If Yes, the process continues to store data in Step 704.

“Wake up threshold” is a static threshold value which is configured in the accelerometer 202. It is used to filter out non-relevant movement and “wakes up” the microprocessor only if a drinking event is detected. This value can be changed by the software inside the microprocessor 201. This threshold can be set for each X, Y and Z axes of accelerometer or for any combination of axes. Also the acceleration direction can be set. This can be set so that the acceleration measurement does not begin until an acceleration parallel the axial direction along the length of the container fulfills the required or preset magnitude. In other words, acceleration measurement begins only if an upward movement of the bottle 102 is detected by accelerometer 202. The magnitude is defined as an absolute value of the acceleration vector (i.e. without directional information) or G-force equivalent.

“Event threshold” is static threshold (i.e., a reference tilt angle) which is configured in the microprocessor 201 during the calibration process by taking into account the physical bottle characteristics input by the user and the measured tilt angles from the calibration period. Event threshold is a threshold which starts and ends the measurement period and it's the first threshold to check if the current event is a drinking event or a non-drinking event. Event threshold can be set by the microprocessor and changed according to the bottle characteristics. Event threshold is a low pass filtered acceleration value also known as a DC value which can be transformed into G-force equivalent or used as a raw acceleration data format. Event threshold value is defined using the formula of acceleration is equal or less than cos(α1)*G (as shown in FIG. 11) where α1 is an empirically determined tilt angle for the corresponding bottle 102 at which angle water starts to drip from the full (i.e., completely filled) bottle when tilting. G is the magnitude of gravitation which is constant and if g-forces are used it is 1 g. When acceleration value is equal to or less than the limit defined by a and G the acceleration measurement is accepted. Preferably, only events between 1 to 10 seconds are considered as drinking events.

“Drinking event duration threshold” is a static threshold which is also configured in the microprocessor 201. It is also defined by acceleration equal or less than sin(α2)*G similar to the Event Threshold but the value of the angle α2 is bigger than α1 as the bottle is more tilted. After the container movement crosses Drinking Event Duration threshold tilt angle of α2, the microprocessor starts calculating how many data points are above the drinking event duration threshold level of α2. The amount of data points above the Drinking Event Duration Threshold α2 is seen as the duration of a drinking event. If the accelerometer sample rate is 20 Hz (i.e., 20 cycles per second) then the microprocessor 201 would need 20 data points above the drinking event duration threshold to count for one second long drinking event. If the time above Drinking Event Duration Threshold is too short or the measurement oscillates around drinking event duration threshold, the event will not be considered a drinking event because duration of drinking would be too short. Determination of duration of a drinking event is explained in more detail in Section 9.2 below.

“Upright threshold” is a value which is used for comparing with a historical record of the bottle's orientations calculated from low pass filtered acceleration data corresponding to acceleration parallel to the bottle and a static threshold value (from within the preferred timeframe of 0 to 1 seconds before the start of the actual measurement as determined by the Event Threshold). To consider the movement a drinking event, this historical data needs to have an acceleration data reading that can be converted into a bottle tilt angle that is close to the value of the Upright Threshold (e.g., 20 degree tilt from the bottle's upright position) before the drinking measurement period begins. (See discussion of Event Threshold in Para 171). If Upright Threshold is not met, the data is not considered drinking event-related.

The sensor band 101, due to its possible symmetrical design, might be inadvertently attached to the bottle in an “upside down” position. As a result, the sensor band 101 can be placed with the accelerometer axis pointing up or down. It can also be turned upside down during usage, since the sensor band can be detached. When the sensor band 101 is attached in an upside down position, the sign of the acceleration from accelerometer 202 will change and the limits need to be adjusted accordingly, e.g. the Wake Up Threshold needs to take into account that the movement “up” now has an opposite sign. Thus, the microprocessor 201 automatically changes the sign of the coordinate system of the accelerometer 202 when the sensor band has been rotated 180-+20 degrees, i.e. upside down, for over a minute.

Step 704: Data is stored to a flash memory 203.

Step 705: Data is transferred wirelessly to a mobile device using rf-transceiver 204.

Step 706: Statistical properties of acceleration data are extracted in the form of features which describe the data in a more compact vector form. This feature vector includes, for example, mean, maximum and minimum values and the frequency domain calculations such as energy.

Calculations are performed inside the hardware (i.e. sensor band 101) with a powerful processor or alternatively in a mobile device or on a cloud server. The same applies to steps 707, 708, 709, 710 and 711.

Step 707: Feature vector is an input for machine learning classification model. The model uses supervised learning model which has been preconfigured using the training data in database. Feature vector and classification are explained in more detail in Section 9.1 below.

Step 708: If the classification output is considered something other than a drinking event, the event information is stored to database 504. If the classification output is considered a drinking event, the process will continue to check if the calibration end condition has been met at Step 709, e.g. or the user giving inputs in the mobile app (e.g. press a button) to end calibration. If calibration end condition has not been met, the event information will be stored to database 504. If the calibration end condition has been met, the process will continue to 710 building the Estimator for the drinking amount.

Step 710: Process of building the Estimator 342 commences by communicating with the database 504 to get the bottle characteristics and sip duration information from previous measurement rounds. Machine learning regression method is used to create a regression model for the Estimator 342 which is stored in database 504. Estimator is explained in more detail in sections 9.4 and 9.5.

Step 709: If the calibration end condition is met, the calibration will end 711 and actual measurement (described in Section 8 below) will start. If the calibration end condition is not met, the calibration process will loop back to step 703 measurement and filtering.

Section 8: Using the Solution

FIG. 8 illustrates of the process of tracking the amount of water consumed from the bottle 102 with the sensor band 101 attached.

Step 801: This step refers to the measurement process during which movement data is recorded, movement data is analyzed and threshold decision made by microprocessor. The steps of the process are as follows: The accelerometer records movement data 8011. The movement data is processed in microprocessor 8012. If the data features do not exceed threshold values 8013 (i.e. the threshold values described in more detail in Step 703 in Section 7 above), the process goes back to await new data from the accelerometer. When it does exceed the threshold values 8013, the process continues through to store data 802.

Step 802: Data is stored to flash memory

Step 803: Data is transferred wirelessly to an app in a mobile device.

Step 804: Statistical properties of acceleration data are extracted in a form of features which describes the data in a more compact vector form.

Calculations are performed inside the hardware (i.e. sensor band 101) with a powerful processor or alternatively in a mobile device or on a cloud server. Same applies to steps 805, 806, 808, 809 and Estimator 342.

Step 805: Classification process (see more detailed description in Section 9.1)

Step 806: If the dataset is classified as a drinking event the process will continue to Estimator 342. Otherwise, the event information will be transmitted to database 504 for storage.

Estimator 342 will take as inputs the duration of the drinking event and the magnitude corresponding to the maximum tilt angle of the bottle during drinking event, and place these into a non-linear/linear function which will give an estimate as a result such as Y=bX+c, (Y=estimate for the drinking amount, b=constant, c=constant and X is duration of the drinking event). The magnitude of the maximum tilt angle value can be used to choose different function for estimating liquid consumed if the maximum tilt angle is very low instead of very steep. In a nutshell, Estimator 342 will estimate the consumed water amount per corresponding drinking event. (See Sections 9.4 and 9.5 for more information about Estimator 342).

Step 808: Estimated consumed water amount will be displayed on a mobile device and stored in the database 504.

Step 809. New and old existing data will be used to update the classification model 805 and the Estimator 342 to better adapt to the user's personal drinking habits.

Section 8A: The Relationship Between Hydration Baserate, Sensor Band, Drinking Event Data And Alerts

In this section we will explain how our technology benefits the user in maintaining a state of balanced hydration.

An embodiment of the inventive process is summarized as follows:

a) Hydration baserate for a user is calculated as described in Section 5.

b) The user drinks from the bottle 102, and sensor band 101 sends the accelerometer data to the mobile app as described in Section 8. From the mobile app the accelerometer data can also be sent to the cloud server 402.

c) In the mobile app or the cloud server 402, the drink amount is estimated using the algorithms as described in Section 8.

d) The drink amount is compared with the computed hydration baserate, and depending on the results, the user receives an alert through the mobile app as a notification and/or sound and/or via the visual indicators (e.g., the Light-Emitting Diodes 205) on the sensor band 101. Our examples in this application describe all the types of alerts in real usage situations.

We are using the following algorithms to assist the user to achieve his hydration goals:

1. [total water intake]=[sip amount 1]+[sip amount 2]+[sip amount 3] . . . etc.

The total water intake is the sum of total sip amounts throughout the day. At the end of each day (at 12 pm midnight), the total water intake goes back to zero, and ready to start tracking the next days' drink amounts.

2. [daily goal]=[FinalBaseRate]

The daily goal for the user is the same as the FinalBaseRate described in Section 5.

3. [awake hours]=[alert end hour]−[alert start hour]

In mobile app settings, the user can configure his wake up time and sleep time. This way the mobile app won't be alarming the user while the user is asleep. For example: The user sets his alert start time at 7 am, since he usually wakes up at that time. The alert end time is set at 11 pm, his sleep time. Thus, the [awake hours] in this case are from [7 am] to [11 pm]=16 hours.

4. [hourly goal]=[daily goal]/[awake hours]

We divide the daily goal by daily awake hours to get an hourly goal amount. This way it is more convenient to follow the progress, and the user does not get alerts all the time or only at the end of the day, when it's too late to act on it. Getting alerts all the time would be annoying, while getting only one alert at the end of the day does not serve the purpose of keeping the user hydrated optimally throughout the day. Optionally, the user can see his hourly goal in the dashboard of the mobile app.

In one embodiment, the mobile app checks the status of hydration every time it receives new data from the sensor band, and also whenever the user opens the mobile app. If the user takes a small sip, of which the amount is less than the hourly goal, the mobile app is set to give an alert after one hour from the previous sip. If the user takes a bigger sip or sips within the next 10 minutes which equal(s) or exceeds his hourly goal, the mobile app is set to give an alert after two hours from the previous sip. In case the user does not drink despite the alert, the alerts will continue to occur in one hour intervals until new drink inputs are received through the sensor band (or alternatively as a manual input, as described in the text explanations related to [302].

When the mobile app “alerts”, it means one or several of the following things happen:
The user receives a push notification on their mobile device's screen
The mobile device gives an audible notification, such as a beep or a tune (can be configured but the user)
The mobile device sends a command via the RF transceiver 204 to the sensor band 101 to start blinking Light-Emitting Diodes 205

The user can use the settings screen 301 in the mobile app to configure which types of alerts they wish to receive.

As mentioned, the alerts can also come through the visual indicators in the sensor band 101. The following presents one possible way to utilize the Light-Emitting Diodes [205] as visual hydration indicators.

LEDs are off: “Hydration situation is good, no need to drink.”

LEDs pulsate slowly: “It has been an hour or more from your last drink, please drink soon.”

LEDs blink rapidly: “It has been over two hours from your last drink, please drink immediately.”

The following is an example describing how the inventive process operates:

John's FinalBaseRate is 2.2 liters per day. At 9 am, he takes a 1.0 deciliter sip, the first of the day, from the bottle. That means his total water intake that day is 1.0 deciliter so far. His hourly goal is 2.0 deciliters The mobile app shows that the same information, his total water intake of 1.0 deciliters, his hourly goal of 2.0 deciliters and his goal for the day, 2.2 liters.

At 10 am, John still has not taken another sip. Thus, the mobile app sends him an alert, which can be in the form a push notification or a visual notification from the sensor band, for example The Light Emitting Diodes 205 can indicate John that it's time to drink. John does not respond, and he gets another alert at 11 am. John takes heed and gulps down a few big sips, totaling 3.5 deciliter. Since this is a bigger amount than his hourly goal, the next time the mobile app gives him an alert is two hours away, at 1 pm.

Section 9: how to Identify a Drinking Event

This section describes how the mobile app 320 distinguishes between a drinking event and a non-drinking event and explains what happens during filtering and classification processes.

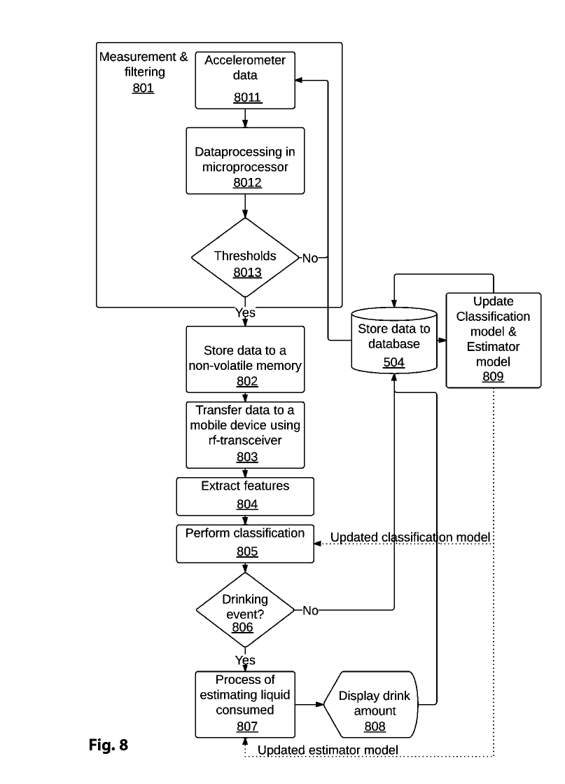

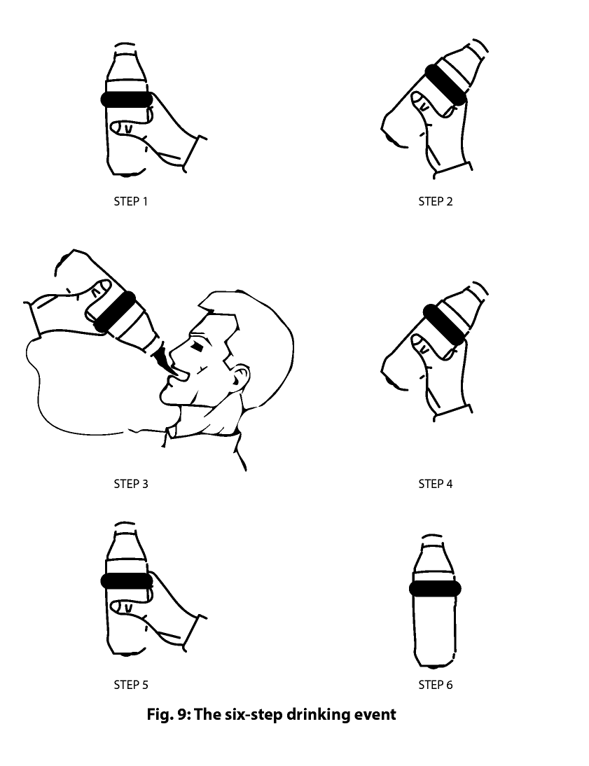

As shown in FIG. 9, the bottle 102 is standing on a table and the sensor band around it is in sleep mode, waiting for activity (Step 1 in FIG. 9). When the user grabs hold of the bottle, the acceleration of the bottle wakes up the microprocessor in the sensor band (Step 2 in FIG. 9). Accelerometer measurement begins when person lifts the bottle and starts tilting it as a drinking movement (Step 3 in FIG. 9). The drinking event is defined as the continuous movement from upright position to a tilted position while drinking, and back to an upright position (Steps 1 to 5 in FIG. 9). Step 6 in FIG. 9 corresponds to a sleep state of the microprocessor 201 when no activity is detected by the accelerometer 202. The bottle 102 can be oriented in any possible orientation in sleep mode. If activity is not detected, the measurement does not start.

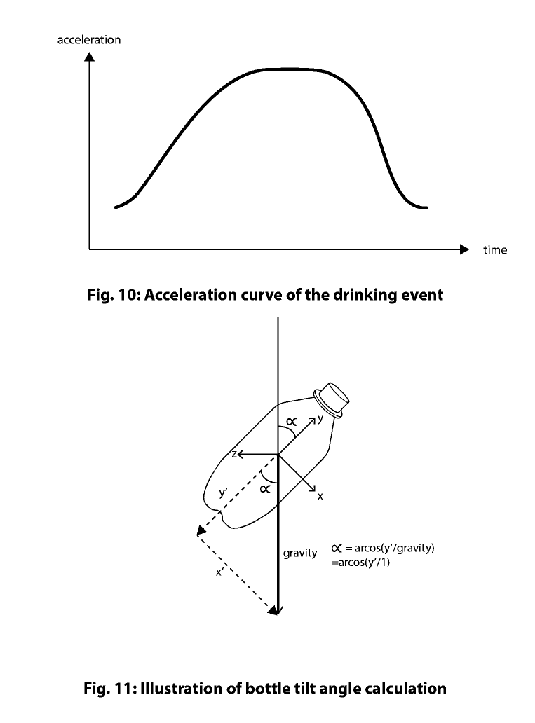

A drinking event can be described as the acceleration curve shown in FIG. 10, where the axis of acceleration is parallel to the height of the bottle.

Since the mobile app 320 does not understand which movement is a drinking event and what is not, the non-drinking events need to be filtered out so that most of the false drinking events are not stored to the flash memory 203 for analysis. To filter out non-drinking events, static threshold values and upright threshold value are used. These are values which correlate or correspond to the acceleration of the bottle and the tilt angle of the bottle 102. Generally, an event that has a duration of less than 1 second and more than 10 seconds is considered a non-drinking event, because the drinking event is considered to take place somewhere between these two values. Duration of a drinking event is calculated to be the time the bottle 102 is tilted more than the Event Threshold limit. During the drinking event the tilt angle of the bottle goes above the Event Threshold and comes back down below the Event Threshold inside the predefined time window of 1 to 10 seconds. If the bottle is tilted above the Event Threshold longer than 10 seconds, the microprocessor 201 of the sensor band 101 will become inactive (i.e. sleep mode) and has to wait for an acceleration value to pass a predefined Wake Up Threshold. When measurement starts, acceleration values are screened so that if the bottle is not in a sufficiently steep tilt angle, the measurement will stop and the event is considered a non-drinking event (i.e., below Event Threshold and Drinking Event Duration Threshold). If the measurement starts in a steep enough tilt angle, but the bottle is, for example, inside a bag and the activity takes enough time to be considered as a drinking event, it must be filtered out by recalling the past orientations of the bottle. For example, if the bottle 102 has not been close to its upright position (e.g., tilted less than 15 degrees off from upright position) during the time period (e.g., one second) immediately prior to measurement (i.e., the last second) it is assumed that bottle 102 is still in the bag and the event is not a drinking event. This is called the Upright Threshold, since it takes into account the historical data of the past orientations of the bottle. If the tilt angle has not been close to upright the current event will not be accepted as a drinking event. The tilt angle α is illustrated in FIG. 11.

If the user takes the bottle 102 from the bag and it is tilted there more than the Event Threshold, the measurement would start. But if the bottle has not been close to upright position during the last second, i.e. the Upright Threshold, the measurement will be cancelled.

However, some datasets will be considered drinking events even though they are not. Sometimes the bottle 102 can rock back and forth inside a bag so that the sensor band 101 will register movement which goes above and comes below the Event Threshold and Drinking Event Duration Thresholds inside the predefined 1-10 second time window. Additionally, 1 second before the movement the bottle has been close to the upright position, so that the Upright Threshold accepts this as a drinking event. This data, even though not representative of a true drinking event, would be considered a drinking event based on these thresholds alone. In order to filter out this kind of data, a machine learning classification method is preferably employed in a device (e.g., a mobile phone) with more computation power than the microprocessor in the sensor band.

9.1 Classification

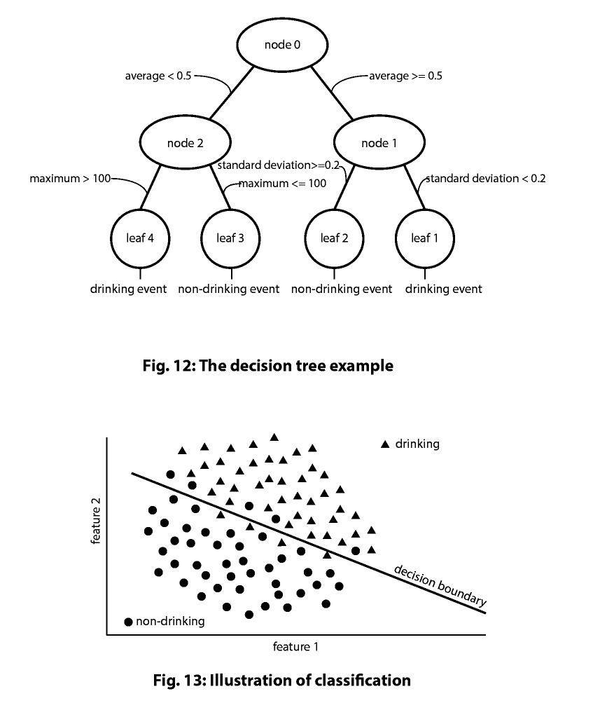

Classification method is used for data that has been already been filtered by the hardware so it will not include events that are considered non-drinking events by the hardware using the aforementioned threshold values. The main purpose of the classification method is to increase the robustness of the detection of drinking and non-drinking events to ensure that the end result for drinking estimation would be as accurate as possible, without non-drinking events causing errors in identification of true drinking events. Classification method such as a decision tree can be taught to classify data into different classes by finding a boundary that divides the data distributions to these classes as well as possible. As shown in FIG. 12, a decision tree is a flowchart-like structure in which an internal node represents a test on an attribute, such as checking the value of standard deviation of the dataset, and if it is below a certain value, it goes to a different node than if the value is above a certain value. Each branch represents an outcome of a test and each leaf node represents a class label such as a drinking or non-drinking event. Classification will terminate at a leaf node which will give the class label. In this example it would indicate whether the event is a drinking or a non-drinking event. A path from the root to a leaf represents classification rules.

Classification is a method where two or more classes of data are separated from each other by finding a decision boundary that divides the classes from each other as well as possible (as illustrated in FIG. 13). The supervised classification model will be first taught by using a learning dataset where the data classes are prelabeled. The learning dataset will consist of feature vectors which describe the data, e.g., the mean and maximum value of the dataset. The dataset consists of the acceleration values or data from all of the axes of the accelerometer resulting from the drinking event (see FIG. 9, step 3). The classification method will find an optimum way of dividing the classes and when the model is ready, a new measurement can be classified by inputting the new feature vector of a new measurement and the model will output the most statistically relevant class label. FIG. 13 illustrates a scatter plot presentation of classification using two different features such as the standard deviation and the mean of the data.

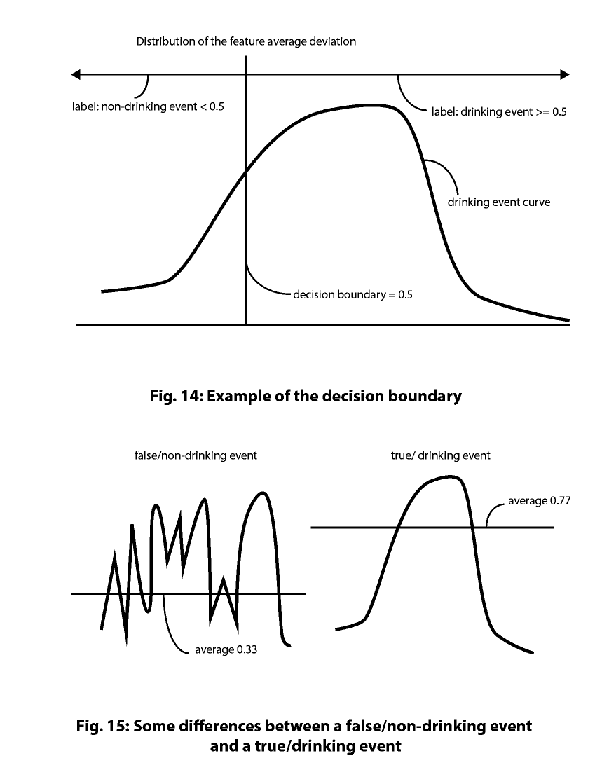

To classify the acceleration data, it needs to be described in statistical terms, such as the mean value of the acceleration and the standard deviation of the acceleration during the activity event. The mean and the standard deviation are called features of the activity event since they describe the data. Decision trees are taught by a learning set which includes features from actual drinking events and features from non-drinking events which have not been filtered successfully by the hardware using the aforementioned threshold values. These datasets are labeled as corresponding drinking and non-drinking events. Decision trees try to find the boundary from the given data that would divide the two classes the best way possible. (FIG. 14).

When the decision trees are taught it can be used to classify new data inputs into false drinking events and true drinking events such as that shown in FIG. 15.

An example of an event classification using a decision boundary: After calculating the feature vector from the event data, an average value could be one of the features. The average could be assigned values between 0 and 5 referring to the steepness of the drinking tilt angle (where 5 means upside down and 0 means an upright position). FIG. 14 illustrates that the decision boundary lies at 0.5 meaning that an event with an average value of less than 0.5 is classified as a non-drinking event and drinking events with average values of more than or equal to 0.5 would be classified as drinking events. So in this example, if an event would have an average value of 0.33 it would be classified as a non-drinking event and would not be used for drink estimation later, since 0.33<0.5.

9.2 Determining Drink Event Duration

When the event is determined to be a drinking event first by the hardware filtering and later by classification, the duration of a drinking event i.e. the length of the dataset above the drinking event duration threshold and the magnitude of the dataset values are used for estimating the amount of liquid drank from the container 102. The magnitude of the dataset values, i.e. steepness of the tilt angle of the bottle can be the low pass filtered maximum value or average of a set of maximum values. See FIG. 16.

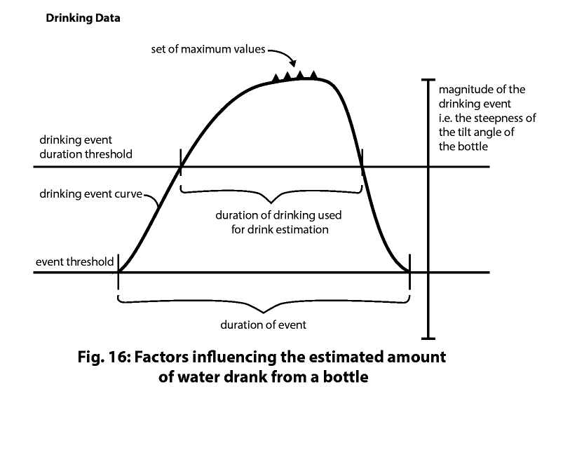

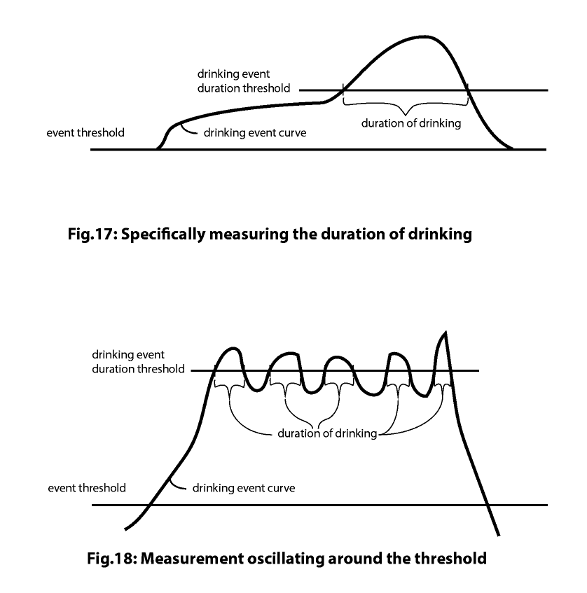

Previously it has been explained that the event is considered a drinking event if the duration of the event is between 1 to 10 seconds which is defined by the Event Threshold. Drinking Event Duration Threshold can be used to set a more specific threshold value for each container type. Instead of setting the whole dataset length as the duration of the drinking event, this Drinking Event Duration Threshold can be a higher value than the Event Threshold, allowing to measure more specifically the duration of a drinking event since the time used for lifting and lowering the bottle part can be reduced (see FIG. 17). This way only data points above the Drinking Event Duration threshold value would be calculated to affect the duration of the drinking event.

Drinking Event duration Threshold can be used to evaluate the data. If the bottle does not tilt enough to cross the Drinking Event Duration Threshold, the measurement could be discarded as non-drinking event. If the measurement oscillates around the Drinking Event Duration Threshold, the measurement could be discarded as a non-drinking event since the person drinking from the bottle would not be able to drink any liquid if this was true since the tilt angle is not steep enough (FIG. 18). The duration of the drinking event is calculated from measurements above the Drinking Event Duration Threshold (see FIG. 17). For example if 20 Hz sample rate is used, twenty datapoints above the Drinking Event Duration Threshold would mean a duration of one (1) second.

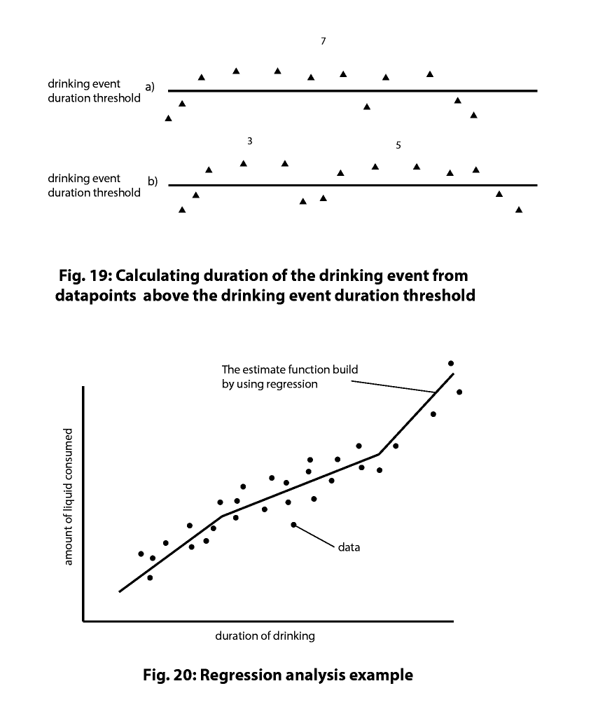

FIG. 19 demonstrates how the duration is calculated. The dataset a) in FIG. 19 gets a value of “7” as duration since it is allowed to go below the Drinking Event Duration Threshold (i.e. represented as the solid black line) without consequences because there can always be an artifact that causes a single datapoint jump in the acceleration for a short period, but it does not mean that the tilt angle actually has changed. The dataset b) gets a value of “3” and “5” for duration since it goes below the Drinking Event Duration Threshold more than once and gets duration value as the number of samples in longest continuous set which are above the Drinking Event Duration Threshold (i.e. represented as the solid black line). In this case the duration is “5”. If the gap between “3” and “5” would be longer these could be considered two separate sips inside the same drinking event. It would mean that the user lowers the bottle a bit between the sips, but the bottle does not cross the Event Threshold to end the event measurement.

As a summary, there are three (3) static threshold values. First one is the Wake Up Threshold set for the accelerometer, which is for the microprocessor of the sensor band 101 to wake up (or activate) and be ready for measurement or to go into sleep mode. Second one is the Event Threshold which is the tilt angle that is considered to be the start of a drinking event, and the time above this threshold needs to be preferably between 1 to 10 seconds in duration. The third one is the Drinking Event Duration Threshold which features an even more tilted bottle angle than the Event Threshold and it is used to calculate more specifically how long the user is drinking, and whether the time beyond the Drinking Event Duration Threshold is short enough for it not to be considered a drinking event. A track competition is a good analogy: there are “ready”, “set”, “go” commands and these static thresholds are pretty much the same. The 1st threshold (i.e., Wake Up Threshold) means ready for measurement, the 2nd threshold (i.e., Event Threshold) starts the measurement and the 3rd threshold (i.e., Drinking Event Duration Threshold) starts the clock and is actually a drinking event as far as the microprocessor is concerned.

9.3 Calibration

User can perform the calibration from the mobile app. The start of the calibration is indicated through user inputs (i.e. pushing a button) in the mobile app. To ensure that the calibration is correct, the user should perform the calibration in a setup where only drinking acts are going to occur and the bottle remains upright on top of table when drinking acts are not occurring. The first and last sip could be indicated by the user themselves by tilting the bottle upside down for a duration of 5 seconds before the first sip and after the last sip. Alternatively the user can input manually to the mobile app before each sip starts. The calibration dataset that is collected is used to build the initial Estimator 342 for the drink amount consumed per sip. This dataset can also be inputted to the classification learning dataset as personalized drink patterns which would help to identify the user's personal pattern. Calibration can also be used to add drinking and non-drinking events to the classification learning set: if the user gets a high amount of false drinking events which show up as false amount of drink consumed, the user could calibrate the device by recording the suspected false event they are doing and labeling it as a non-drinking event. For example if running causes false readings the user could choose to add non-drinking event in the mobile app and then collect data for example for a minute. After that the classification algorithm will attempt to include this data to a learning set and change the classification to reject this kind of bottle movement as a non-drinking event.

9.4 Regression and the Estimator

A regression means fitting a curve to a dataset, as shown in FIG. 20. Here we use regression as a way to fit a curve to data and use the function of the fitted curve to estimate the new input data values by placing data to the function and calculating the result. In this implementation the regression curve will be fitted to the drinking event data so that the x,y coordinates consist of the duration of the measured drinking event and the estimate of amount of liquid consumed during that sip. The regression gives a function to which the new drinking event duration inserted will give an estimate for the amount of liquid consumed.

The initial estimates of the amount of liquid consumed can be calculated from the calibration dataset as follows, but is not restricted to the following.

Amountpersips_vector=Σ_(n=1)̂kΣ_(m=1)̂l(((sip(m)_length+w_{—}m1)/(total_length+w_{—}m2))*container_volume)

Where sip(m)_length is the duration of a measured sip m. w_m1 is gaussian noise added to sip(m)_length and it can be either plus or minus-signed. The total length is the total duration of measured sips by summing all the sip(m)_length. w_m2 is gaussian noise added to total_length and can be different than w_m1. container_volume is the volume of the container. This calculation is done k times so that we get k different values for each sip. To reduce the bias of the naive assumption, noise variables can be introduced.

By updating this initial regression curve with new data points e.g. recalibrating (duration and consumption estimate) will give a better estimate in the future. If possible, the most accurate estimate could be done if real and measured drink amounts could be inserted e.g. by using a scale in the calibration phase.

9.5 Example Case of Building an Estimator Using Calibration Data:

In calibration phase an Estimator 342 is built. We can get an example (FIG. 21) for Estimator 342, if the data is fitted only once without adding the noise. When the volume of the bottle is known e.g. 500 ml, duration of each sip and the total duration of sips can be calculated. For example sip1=3 s, sip2=4 s, sip3=2 s, sip4=6 s, sip5=4 s and total=19 seconds

point1=3/20*500=75

point2=4/20*500=100

point3=2/20*500=50

point4=6/20*500=150

point5=5/20*500=125

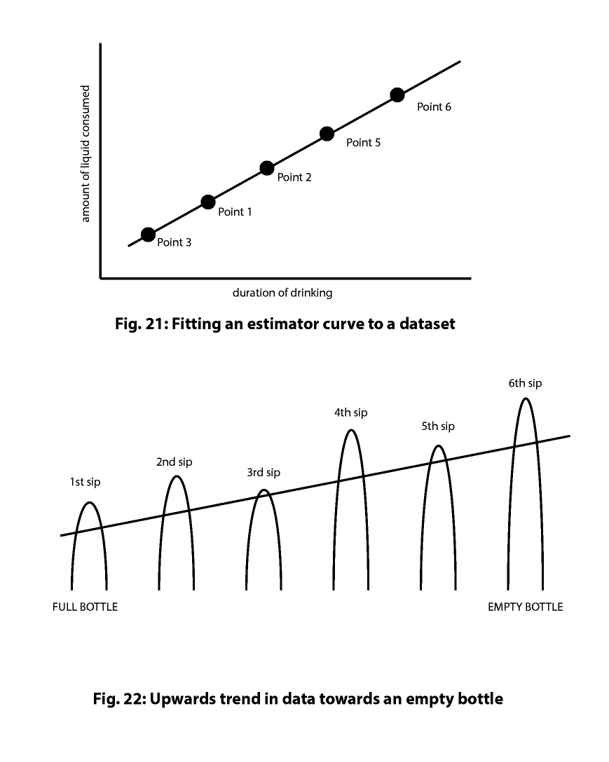

The Estimator 342 is the fitted curve to datapoints previously mentioned in a form of y=bX+C (FIG. 21). In this example the estimate for water consumed (Y) would be Y=25*X+0 where X is time in seconds. When new drink duration information is inputted in the Estimator function, an estimate for the water amount consumed is received. Usually when the bottle is full, more water is drank in less time than when bottle is close to empty. The magnitude of acceleration data, i.e. the maximum tilt angle, can be used to estimate when the bottle is full, since the user needs to tilt the bottle less to be able to drink when the bottle is full compared to when the bottle is almost empty. This also impacts the duration of drinking since it takes less time to finish drinking when the bottle is full, as compared to when the bottle is almost empty. This is because the user needs to tilt more to drink and for this longer movement to cover greater angular distance takes more time, even though the actual drink amounts would be equal in both situations. This imbalance between the first and last sip can be adjusted by reducing the estimated drink amount of the last sip and increasing the estimated drink amount of the first sip. There is also an upward trend (see FIG. 22) caused by the bottle tilt angle increasing towards an empty bottle. This can help to estimate when container is close to empty, but it is not always obvious and usable.

Section 10: Use Case Example Of Jack, The Cycling Enthusiast

This story represents one possible use case for our solution. The storyline is in italic to distinguish it properly from the system descriptions.

Jack loves road biking. It's Saturday morning, and Jack is preparing to go for a bike ride in the afternoon. Jack mounts his bike on the roof racks of the car and goes back in the house to pack the rest of his gear in his trusty gym bag. Among other things, he needs to stay properly hydrated before, during and after the race so he fills up two water bottles. The first one is an aluminum bottle with a regular twist-cap and the second one is a plastic, squeezable bike bottle with a nozzle type mouthpiece. He tosses the bike bottle in his bag. Jack slips the sensor band 101 around the aluminum bottle, and opens the mobile app 401 on his mobile phone. He opens the settings screen 301, checks that the basic information he has previously inputted is still accurate (luckily, he has not gained weight!) and also links the mobile app 401 with his latest App Store purchase, a cycling app called Strava. The hydration baserate calculation and its adjustment with third-party data is explained in more detail in Section 5.

Before tossing the aluminum bottle in his gym bag also, Jack opens the cap and takes a sip from the bottle. The initial bottle movement shaking and movement up wakes up the sensor band 101 since the Wake Up threshold will be exceeded. When the bottle tilt angle crosses the Event Threshold value the sensor band 101 starts to record the event, and when the Drinking Event Duration Threshold value is exceeded by the increasing tilt angle of the bottle the microprocessor 201 starts recording the duration of the drinking event. When the user lowers the bottle after taking a sip, the Event Threshold has been crossed so that the tilt angle is less than the Event Threshold. Then the sensor band 101 will store the acceleration data from all the available axis X, Y and Z of the accelerometer to a flash memory with the information of the duration of the drinking event provided that the duration of the event is between 1 second to 10 seconds; if the duration of the event falls outside this time window, the sensor band 101 will not store the data to the flash memory. Using a sample rate of 20 Hz, this would mean that data would consist of 20 samples for each axis per second. The sensor band 101 has an RF-transceiver 204 which allows it to communicate with other devices such as a mobile device for example. The mobile device which has the mobile app can then “inquire” for the data from the sensor band using wireless communication means, such as Bluetooth. When the sensor band 101 receives a query for the stored data, it sends the data to the mobile device. This stored data is already filtered and determined to be relevant drinking event data which consists of events within a predetermined time window such as 1 to 10 seconds. The mobile app performs a classification task to further ensure that the data is a true drinking event and not a false drinking event. Since the event was a drinking event, the mobile app uses the duration of the sip to estimate the amount of liquid consumed from the bottle by inputting the duration data to the Estimator 342 that utilizes a function such as a linear curve which is predefined from the calibration data. This estimate for amount of liquid consumed is used by the mobile app to display to the user how much they drank. More details of the above described process can be found in Section 8 of this specification.

After the sip, he takes a look at the mobile app, and notices that it has received information from his sip. It's a warm day and the mobile app suggests that the optimal hydration rate for Jack today is 2.2 liters. Great, good to know. Jack then puts the mobile device in his pocket. The mobile app screens and main functions are described in more detail in Section 3 and the temperature adjustment of hydration baserate in Section 5 of this specification.

Jack grabs the gym bag and walks 300 meters to his car, and places the bag in the trunk. He grabs the aluminum bottle (it has the sensor band around it) from the bag and goes to sit on the driver's seat. It's a two-hour drive to get to the bicycling start venue, and he wants to have his water bottle at hand since starting the biking properly hydrated is very important. However, his mother calls him on the phone when he starts driving, and she just won't stop talking Jack forgets about the water bottle. Luckily, after about an hour, the mobile app alerts and he sees a push notification from the mobile app on the screen. Jack grabs the bottle and takes a few long sips. The relationship between the hydration baserate, sensor band data and alerts is explained in detail in Section 8A of this specification.

In a while Jack arrives to the place where he starts his bike tour. Jack leaves the aluminum bottle in the car but removes the sensor band first. He grabs the bike bottle from the gym bag and slips the sensor band around it. It's very convenient for Jack that the sensor band 101 is designed to work with different bottle shapes, sizes and materials and that changing it from one bottle to another is very effortless.

Jack puts the bike bottle on the bottle holder which is attached to the down tube of his bike. His smartphone goes on a holder attached to the bike handlebar. This way he can keep an eye on the route, see cycling-related information from his new Strava cycling app and also follow his hydration goals from the mobile app 303. Jack starts his bike tour, which goes around a 40-mile route along the roads. Some of the roads are in quite bad condition, though, and he has to be aware of potholes and endure occasional tremble caused by the uneven roads. Since the bottle encounters acceleration and tilt angle changes which could be causing drinking events that are false, there needs to be a system in place to remove these false drinking events. This system consists of two layers. First layer which is called filtering happens inside the hardware. The second layer is called classification, and in most cases that happens in a more powerful processing domain such as a mobile device with the data that the hardware has already filtered to be relevant. If Jack places the bottle to a holder which has the tilt angle greater than the event and drinking event duration thresholds the sensor band will start measurement, but will stop after 10 seconds has passed. Thus, this data is discarded as non-drinking event and it will not be stored into the flash memory. If the bottle happens to tilt back below the Event Threshold value before 10 seconds has passed this data would be considered a drinking event, but only if the bottle has been close to upright position inside a 1 second time window before the measurement was triggered. The Upright Threshold compares the history which holds information about the past orientations of the bottle and considers an event a drinking event if and only if the bottle has been above the Upright Threshold value which is close to the bottle being upright position (within +/−15 degrees of upright position) in the time period (e.g., last second) prior to measurement. Threshold values filter events when carrying the bottle inside a bag, but sometimes the bottle happens to be close to upright position before the measurement starts. The movement is accepted by the threshold values as a drinking event since acceleration of the bottle and thus bottle tilt angles resembles movements seen in FIG. 9 and the duration is between 1 to 10 seconds. This could happen e.g. when Jack is rocking a bag from side to side with the bottle inside. To remove this kind of false drinking events the classification task is performed (e.g. in the mobile device). Even though the data resembles the actual drinking event it has different characteristics, such as bigger standard deviation or lower average value. The classification method uses the characteristics which are called features to check that which event the data resembles more; a non-drinking event or a drinking event. In this case the classification would label the event a non-drinking event and it would not be considered as a drinking event. The filtering and classification processes are described in more detail in Section 8A of this specification.

Biking is a tough sport, and Jack's new biking app is tracking his caloric expenditure. Luckily, this caloric consumption information is also utilized by the mobile app, which adjusts Jack's need for hydration accordingly. This way Jack knows how much to drink in order to stay properly hydrated, even during sports activities. The hydration baserate adjustment with third-party caloric data is explained in more detail in Section 5 of this specification.

After a while, Jack is done with his bike tour. He packs his gear in the car, but before taking the driver's seat, he removes the sensor band 101 from the bike bottle and slips it around the aluminum bottle he left in the car front seat. He puts the radio on, amps up the volume and starts to listen to his favorite show. After an hour, he notices the lights on the sensor band 101 start blinking Jack has been so immersed in the radio show that he has forgotten to drink again, and the loud volume has prevented him from hearing the alerts, the audible beeps on his mobile phone. The LED visual indicators and audible notifications are explained in more detail in Section 8A of this specification.

It was a good ride, and for the rest of the day Jack relaxes at home. He has refrigerator-cold water with lemon wedges in the fridge, and now he slips the sensor band 101 around a tall glass that he fills up every now and then from a carafe. It is contemplated that the sensor band 101 works on other containers other than bottle-shaped vessels.

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

What is claimed is:

1. A computer-implemented method of determining fluid intake by a user from a fluid container containing liquid, comprising the steps of:
receiving, by a computer processor, physical characteristics of the fluid container including its shape and volume;
measuring, by an accelerometer, movement of the fluid container;
processing acceleration data generated by the accelerometer during movement of the fluid container;
determining and monitoring, by the computer processor, a tilt angle of the fluid container during movement of the fluid container based on the acceleration data;
determining the movement as a true or false drinking event based on parameters including the determined tilt angle of the fluid container and duration of the movement;
determining an amount of reduction of fluid from the fluid container based on the tilt angle, the duration of the movement, and the physical characteristics of the fluid container when the movement is determined as a true drinking event; and
outputting a signal indicative of the reduction of fluid from the fluid container.

2. The method of claim 1, further comprising the steps of:
storing the amount of reduction of fluid from the fluid container over a period of time;
receiving, by the computer processor, personal information including age, gender, and/or weight of the user;
determining a base rate of hydration required by the user based on the personal information; and
comparing the base rate of hydration of the user with the amount of reduction of fluid from the fluid container over the period of time.

3. The method of claim 2, further comprising the step of indicating to the user a result of the step of comparing.

4. The method of claim 2, further comprising the steps of determining an amount of deficit of fluid required by the user from the step of comparing, and indicating to the user the amount of deficit.

5. The method of claim 1, further comprising the steps of receiving by the computer processor ambient temperature data from a third party server and adjusting the base rate of hydration of the user based on the ambient temperature data.

6. The method of claim 1, further comprising the steps of:
defining in the computer processor an Event Threshold criterion for defining a first reference tilt angle of the fluid container at which fluid flows out of the fluid container, and which is used for starting and ending measurement by the accelerometer; and
defining an Upright Threshold criterion for defining a second reference tilt angle of the fluid container at which the fluid container is in an upright position, and which is used for comparing a historical record of tilt angles of the fluid container determined from the acceleration data, and for determining when the fluid container is in its upright position before measurement of a drinking event can start.

7. The method of claim 6, further comprising defining in the accelerometer a Wake-up Threshold criterion for filtering the acceleration data from the accelerometer and for activating the computer processor only if the acceleration data exceeds the Wake-up Threshold.

8. The method of claim 6, further comprising the step of defining in the computer processor a Drinking Event Duration Threshold criterion for defining a second reference tilt angle of the fluid container for defining the determined movement as a true drinking event.

9. The method of claim 7, further comprising the step of activating the computer processor when the acceleration data satisfy the Wake Up Threshold criterion.

10. The method of claim 6, further comprising the step of determining whether the determined tilt angle of the fluid container satisfies the Event Threshold criterion.

11. The method of claim 8, further comprising the step of determining whether the determined tilt angle of the fluid container satisfies the Drink Event Duration Threshold criterion.

12. The method of claim 6, further comprising the step of determining whether the determined tilt angle of the fluid container satisfies the Upright Threshold criterion.

13. The method of claim 1, wherein the step of determining the movement uses a classification method including a decision tree to divide the acceleration data into a first class of data representing drinking events and a second class of data representing non-drinking events.

14. The method of claim 13, wherein the step of determining the reduction of fluid from the fluid container includes a step of estimating the reduction of fluid from the fluid container based on the first class of data for drinking events.

15. The method of claim 14, wherein the step of estimating uses a linear equation to fit the first class of data.

16. An apparatus for determining fluid intake by a user from a fluid container containing liquid, comprising:
an accelerometer for generating acceleration data based on movement of the fluid container;
a microprocessor for processing the acceleration data and for computing a tilt angle of the fluid container based on the acceleration data;
a user interface module for receiving personal information and physical characteristics of the fluid container from the user and for outputting data to the user;
a classification module for classifying the acceleration data from the accelerometer into drinking and non-drinking events;
an estimator module for determining an amount of fluid reduction from the fluid container based on the classified acceleration data from the classification module and the physical characteristics of the fluid container; and
a water baserate calculation and optimization module for computing a baserate of hydration of the user based on the personal information received from the user interface and comparing the baserate with the amount of fluid reduction determined by the estimator module.

17. The apparatus of claim 16 further comprising an indicator light and/or visual information screen connected to the user interface module.

18. The apparatus of claim 16, further comprising a memory chip for storing the acceleration data.

19. The apparatus of claim 16 further comprising a Feature Vector Calculation Module.

20. The apparatus of claim 16 further comprising a Calibration Module.
